(19) United States
(12) Patent Application Publication
(10) Pub. No.: US 2026/0069836 A1
(43) Pub. Date: Mar. 12, 2026

(54) INFLATION ADJUSTMENT ATTACHMENT DEVICE FOR PRECISE MEDICAL DEVICE DEPLOYMENT AND METHODS RELATING TO THE SAME

(71) Applicant: MiRus LLC, Marietta, GA (US)

(72) Inventors: Alex Panter, Marietta, GA (US); Jason White, Atlanta, GA (US); Jay Yadav, Atlanta, GA (US); Jorge Jimenez, Marietta, GA (US)

(21) Appl. No.: 19/316,122

(22) Filed: Sep. 2, 2025

Related U.S. Application Data
(60) Provisional application No. 63/691,372, filed on Sep. 6, 2024.

(51) Int. Cl. A61M 25/10; A61F 2/24; A61F 2/958

(52) U.S. Cl. CPC A61M 25/10182; A61F 2/2433; A61F 2/958; A61M 25/10184; A61M 25/104; A61M 2205/3379; A61M 2205/581; A61M 2205/582; A61M 2205/584

(57) ABSTRACT

Inflation adjustment attachment devices, systems, and methods for accurately deploying a medical device. The inflation adjustment attachment devices, systems, and methods are configured to allow for the controlled increase of the balloon volume during inflation of the prosthetic heart valve, and thereafter without having to deflate the balloon, enable an additional volume to be added to the inflation device.

### REFERENCED APPLICATIONS

The present application claims priority on U.S. Patent Application Ser. No. 63/691,372 filed Sep. 6, 2024, which is fully incorporated herein by reference.

### FIELD OF DISCLOSURE

The present disclosure relates generally to inflation adjustment attachment devices and methods for the precise deployment of a medical device, and more particularly to inflation adjustment attachment devices and methods for the precise deployment of an expandable medical device in a treatment area, and even more particularly to inflation adjustment attachment devices and methods that allow a user to incrementally increase inflation volume of a balloon and further expand a prosthetic heart valve, stent, angioplasty balloon, or other expandable medical device, without having to deflate the balloon, and add additional volume to the inflation device, and perform post dilation of the valve with the additional volume.

### BACKGROUND OF DISCLOSURE

Medical devices such as transcatheter aortic valves (TAVs) represent a significant advancement in prosthetic heart valve technology. A TAV is generally designed to be remotely placed within a patient's diseased aortic valve to take over the function of the native valve. TAVs bring the benefit of heart valve replacement to patients that would otherwise not be operated on, as transcatheter aortic valve replacement (TAVR) can be used, for example, to treat aortic valve stenosis in patients who are classified as high-risk for open heart surgical aortic valve replacement (SAVR). Major TAVR advantages to the traditional surgical approaches include refraining from cardiopulmonary bypass, aortic cross-clamping and sternotomy which significantly reduces patients' morbidity.

Many collapsible/expandable stents, heart valves, TAVs, and the like are inserted into a patient via the patient's vascular system and then expanded at the treatment site. Typically, these stents and valves are initially crimped onto a deflated balloon of a balloon catheter or similar tube-like device. Once the balloon (and the crimped valve) is positioned at the predetermined treatment site within the patient, the balloon is inflated via a fluid displacement device, syringe, or similar inflation device to correspondingly expand the stent prosthetic heart valve and lodge the stent or prosthetic heart valve at the treatment site.

Conventional methods in the art typically include forcing a fluid (or gas), such as a saline solution, through the balloon catheter and into the balloon. As the balloon begins the fill with the fluid, the balloon increases in volume and correspondingly expands the stent or prosthetic heart valve to a target diameter and/or size. The target diameter/size of the stent or valve is generally determined for each patient as an outcome of pre-procedure planning (e.g., imaging, electrocardiogram testing, etc.) and patient diagnostic information. The inflation device, which is in fluid connection with the balloon catheter, is typically prepared/filled with a predetermined amount of inflation fluid based on the predetermined target diameter and/or size of the stent or valve. To manually inflate the balloon, the user advances an inflation plunger/piston of the inflation device until it stops, indicating that the full amount of the inflation fluid has been delivered to the balloon and that the predetermined target diameter/size of the stent or valve has been achieved.

Several complications and drawbacks are associated with such inflation device systems and methods. For one, the initially targeted deployed valve size/diameter is generally less than the potentially optimal deployed stent or valve size/diameter, in order to reduce the risk of complications that could result from stent or valve oversizing. However, current inflation devices allow for only one fill volume. Accordingly, if the user/physician decides that additional inflation fluid and/or a larger stent or valve size/diameter is necessary, the user/physician needs to deflate the balloon, add an additional amount of liquid to the inflation device, and then perform an additional post dilation of the stent or valve with the additional amount of liquid. These additional steps increase the overall time of the procedure and increase the risk for vascular complications such as severe bleeding, sudden cardiac death, thromboembolic events, etc. Further, each inflation/deflation of the balloon increases the risk of thromboembolic events and damage to the stent or leaflets and/or frame of the prosthetic heart valve.

As such, there is an ongoing need for inflation adjustment attachment devices (e.g. clips, rings, ring clips, C-clips, latches, fasteners, tabs, clasps, pins, cotter pins, etc.) and methods that couple/attach/fasten to inflation devices and allow for the incremental increase of the balloon volume during first inflation of the stent or prosthetic heart valve, without having to deflate the balloon, add an additional volume to the inflation device, and perform post dilation of the stent or valve with the additional volume. In some non-limiting embodiments, the disclosed inflation adjustment attachment devices can advantageously include one or more audible mechanisms that are configured to provide feedback when: (i) the inflation target of the balloon is achieved; (ii) the size/diameter target of stent or prosthetic heart valve is achieved; (iii) the balloon volume is less than the inflation target; (iv) the stent or prosthetic heart valve size/diameter is less than the target; (v) the balloon volume is greater than the inflation target; and/or (vi) the stent or prosthetic heart valve size/diameter is greater than the target. The disclosed inflation adjustment attachment devices and methods address other deficiencies of prior art inflation devices, systems, and methods.

### SUMMARY OF THE DISCLOSURE

The present disclosure relates to inflation device inflation adjustment attachment devices and methods for the precise deployment of a medical device, and more particularly to inflation adjustment attachment devices and methods for the precise deployment/expansion of a stent, prosthetic heart valve or angioplasty balloon, and even more particularly to inflation adjustment attachment devices (e.g. clips, rings, ring clips, C-clips, latches, fasteners, tabs, clasps, pins, cotter pins, etc.) methods that allow a user to incrementally increase inflation volume of a balloon and further expand a stent, or prosthetic heart valve or angioplasty balloon, without having to deflate the balloon, add additional volume to the inflation device, and perform post dilation of the stent, valve or angioplasty balloon with the additional volume. Although the present disclosure will particularly discuss inflation adjustment attachment devices and methods for use with prosthetic heart valves, it will be appreciated that the inflation adjustment attachment devices and methods in accordance with the present disclosure can be used with other types of balloon or fluid expandable medical devices (e.g., stents, angioplasty balloon, etc.) that are expanded within a patient at a predetermined treatment area.

In one non-limiting aspect of the present disclosure, there is provided a method for accurately deploying a medical device, the method comprising the steps of: providing an inflation device that is configured to inflate a balloon and correspondingly expand a medical device; preparing the inflation device with a predetermined maximum amount of a substance (e.g., fluid, etc.) to inflate the balloon or otherwise expand the medical device to a predetermined size; coupling a first inflation adjustment attachment device having a predetermined thickness to the inflation device, wherein the first inflation adjustment attachment device is configured as a first stop that allows for incremental inflation of the balloon; activating/using the inflation device to administer a first volume of the substance to the balloon; deactivating the inflation device when the first inflation adjustment attachment device is engaged; and subsequent to deactivating/terminating use of the inflation device, uncoupling the first inflation adjustment attachment device from the inflation device if additional fluid is required for inflation of the medical device, and re-activating/again using the inflation device to administer a retained volume of the substance to the balloon, wherein the retained volume of the substance substantially corresponds to the predetermined thickness of the first inflation adjustment attachment device.

In another and/or alternative non-limiting aspect of the present disclosure, the method optionally further comprises the steps of: coupling a second inflation adjustment attachment device along with the first inflation adjustment attachment device, and wherein the second inflation adjustment attachment device has a predetermined thickness to the inflation device (which predetermined thickness can the same or different from the first inflation adjustment attachment device), wherein the second inflation adjustment attachment device is configured as a second stop that allows for incremental inflation of the balloon/fluid expandable device after the first inflation adjustment attachment device has been removed from the inflation device; subsequent to re-activating/again using the inflation device, deactivating/terminating use of the inflation device when the second inflation adjustment attachment device is engaged and uncoupling the second inflation adjustment attachment device from the inflation device; and re-activating/again using the inflation device to administer a second retained volume of the substance to the balloon/fluid expandable device, and wherein the second retained volume of the substance substantially corresponds to the predetermined thickness of the second inflation adjustment attachment device. As can be appreciated, additional inflation adjustment attachment devices can be used with the inflation device (e.g., third, fourth, fifth, etc. inflation adjustment attachment devices).

In another and/or alternative another non-limiting aspect of the present disclosure, the predetermined thickness of the first inflation adjustment attachment device equals the predetermined thickness of the second inflation adjustment attachment device; or the predetermined thickness of the first inflation adjustment attachment device differs from the predetermined thickness of the second inflation adjustment attachment device. The thickness of each of the inflation adjustment attachment devices can correspond to a certain amount of additional fluid (e.g., 0.01 ml, 0.1 ml, 0.5 ml, 1 ml, 2 ml; 0.01 ml to 10 ml and all values and ranges therebetween) that can be applied to the medical device when the inflation adjustment attachment device is removed from the inflation device.

In another and/or alternative another non-limiting aspect of the present disclosure, the inflation device comprises a housing that is configured to receive a substance (e.g., fluid, water, saline solution, drug solution, etc.); and a plunger having a shaft and a handle, wherein the plunger is at least partially disposed within the housing. In one non-limiting aspect of the present disclosure, each of the inflation adjustment attachment devices optionally comprise a body having a channel formed therein that is configured to partially or fully receive the shaft of the plunger (e.g., partially or fully encircle the shaft of the plunger, etc.). In one non-limiting aspect of the present disclosure, one or more of the inflation adjustment attachment devices optionally are clips, ring clips, C-clips, clasps, tabs, or combinations thereof. In another non-limiting embodiment, one or more of the inflation adjustment attachment devices optionally includes a grasping arrangement (e.g., ring, flange, loop, etc.) that facilitates in the grasping of a portion of the inflation adjustment attachment device by a user so the user can conveniently insert the inflation adjustment attachment device on the plunger, and/or to remove the inflation adjustment attachment device from the plunger when such removal is desired.

In another and/or alternative non-limiting aspect of the present disclosure, the one or more inflation adjustment attachment devices optionally comprise an interactive element in communication with a latch, wherein activation of the interactive element causes the latch to disengage from the plunger and the shaft to travel within the housing of the inflation device. In one non-limiting aspect of the present disclosure, the interactive element is optionally a button, a knob, a switch, a lever, a grip, a controller, a toggle, or combinations thereof. In one non-limiting aspect of the present disclosure, the one or more inflation adjustment attachment devices optionally are clips, clasps, tabs, or combinations thereof.

In another and/or alternative non-limiting aspect of the present disclosure, one or more of the first inflation adjustment attachment devices optionally includes a pin that is configured to engage (e.g., removably engage, etc.) an aperture formed in the shaft of the plunger. In one non-limiting aspect of the present disclosure, the one or more inflation adjustment attachment devices optionally includes a cotter pin.

In another and/or alternative non-limiting aspect of the present disclosure, one or more of the inflation adjustment attachment devices optionally further comprise one or more audible and/or haptic mechanisms that are configured to provide real-time feedback of the amount of the fluid presently delivered to the balloon.

In another and/or alternative non-limiting aspect of the present disclosure, one or more of the inflation adjustment attachment devices optionally includes a visual indicator (e.g., color, shape, symbol, number, etc.) that provides visual information to a user as to the amount of fluid that the inflation adjustment attachment device will allow the inflation device to insert into the balloon or into at least a portion of the medical device when the inflation adjustment attachment device is removed from the inflation device (e.g., plunger shaft, etc.). In one non-limiting embodiment, one or more of first inflation adjustment attachment devices optionally includes identifying indicia that provides the retained volume and/or the second retained volume of fluid associated with the predetermined thicknesses of the inflation adjustment attachment device. In another non-limiting embodiment, one or more of the inflation adjustment attachment devices are optionally color coded to indicate the retained volume of fluid associated with the predetermined thicknesses of the inflation adjustment attachment device.

In another and/or alternative non-limiting aspect of the present disclosure, the fluid in the inflation device is a liquid (e.g., water, saline solution, drug solution, etc.) or a gas (e.g., air, nitrogen oxygen, argon, etc.).

In another and/or alternative non-limiting aspect of the present disclosure, the medical device optionally is an expandable stent or an expandable frame of a prosthetic valve (e.g., prosthetic heart valve, etc.).

In another and/or alternative non-limiting aspect of the present disclosure, there is provided a first inflation adjustment attachment device for use with an inflation device, wherein the first inflation adjustment attachment device comprises an arrangement to couple the inflation adjustment attachment device to a plunger shaft or other portion of the inflation device, and wherein the inflation device is configured to a) inflate a balloon and correspondingly expands at least a portion of a medical device or b) expands at least a portion of a medical device; and a predetermined thickness of the first inflation adjustment attachment device substantially corresponds to a retained volume of a substance within the inflation device, and wherein the inflation device administers a first amount of fluid to the balloon or medical device until the first inflation adjustment attachment device inhibits or prevents further advancement of the plunger in the housing, and wherein the first inflation adjustment attachment device is optionally uncoupled from the plunger shaft or other portion of the inflation device, and the inflation device is thereafter re-activated/again used to administer at least a portion of the retained volume of the fluid in the inflation device to the balloon or medical device after the first inflation adjustment attachment device is uncoupled from the plunger shaft or other portion of the inflation device. In one non-limiting, embodiment, after the first inflation adjustment attachment device is uncoupled from the plunger shaft or other portion of the inflation device, a second inflation adjustment attachment device can optionally be coupled to the plunger shaft or other portion of the inflation device. The second inflation adjustment attachment device can optionally have a thickness that is the same or different (e.g., less, etc.) than a thickness of the first inflation adjustment attachment device such that when the second inflation adjustment attachment device is coupled to the plunger shaft or other portion of the inflation device, the plunger can be advanced by the inflation device administers so that a second amount of fluid is inserted into the balloon or medical device until the a portion of the plunger shaft (e.g., handle of the plunger shaft, etc.) is prevented from further advancement in the housing by the second inflation adjustment attachment device. As can be appreciated, these optional steps can be repeated for a third, fourth, etc. inflation adjustment attachment device (e.g., third inflation adjustment attachment device has a thickness that is the same or different (e.g., less, etc.) than the second inflation adjustment attachment device, the fourth inflation adjustment attachment device has a thickness that is the same or different (e.g., less, etc.) than the third inflation adjustment attachment device, etc.) so as to controllably insert incremental amount of fluid into the balloon or medical device until the desired volume of fluid has been inserted into the balloon or medical device.

In another and/or alternative non-limiting aspect of the present disclosure, there is provided an inflation system that is configured to accurately deploy a medical device, wherein the inflation system comprises an inflation device for inflating a balloon coupled to a medical device or to at least a portion of a medical device, and wherein the inflation device comprises a housing that is configured to receive an inflation substance; and a plunger having a shaft, wherein the plunger is at least partially disposed within the housing; and a first inflation adjustment attachment device that has a predetermined thickness that is configured to control/limit an amount of the inflation substance delivered to the balloon or to at least a portion of the medical device when the first inflation adjustment attachment device is a) positioned on the inflation device, and/or b) when the first inflation adjustment attachment device is removed from the inflation device, and wherein the first inflation adjustment attachment device is releasably coupled to the plunger, wherein the plunger is configured to be advanced in the housing of the inflation device to administer a first volume of the substance to the balloon or to at least a portion of the medical device until the first inflation adjustment attachment device stops the advancement of the plunger in the housing such that the plunger is prevented from further advancement in the housing of the inflation device, and wherein the first inflation adjustment attachment device is optionally uncoupled from the shaft or other portion of the plunger, and thereafter a) the shaft of plunger can be further advanced in the housing of the inflation device to administer at least a portion of a retained volume of the substance in the housing to the balloon or to at least a portion of the medical device, or b) a second inflation adjustment attachment device is coupled to the shaft or other portion of the plunge that has the same or different thickness (e.g., a smaller thickness, etc.) than the first inflation adjustment attachment device, and wherein after the second inflation adjustment attachment device is coupled to the plunger, the plunger can be further advanced in the housing of the inflation device to administer at least a portion of a retained volume of the substance in the housing to the balloon or to at least a portion of the medical device until the movement of the plunger is stopped by the second inflation adjustment attachment device, and wherein the volume of the substance that is administered to the balloon or to at least a portion of the medical device after a) the first inflation adjustment attachment device is uncoupled from the shaft or other portion of the plunger and the plunger is advanced in the housing of the inflation device, and/or b) after the first inflation adjustment attachment device is uncoupled from the shaft or other portion of the plunger, and the second inflation adjustment attachment device is coupled to the shaft or other portion of the plunger and the plunge, and the plunger is thereafter advanced in the housing until the second first inflation adjustment attachment device stops the advancement of the plunger in the housing, corresponds to the predetermined thickness of the first or second inflation adjustment attachment devices.

One non-limiting object of the present disclosure is the provision of an inflation adjustment attachment device and a method that enables a user to control and/or to incrementally increase inflation volume of a balloon or a portion of a medical device so as to controllably expand the medical device without having to deflate the balloon or otherwise withdraw fluid from the balloon or medical device so as to add additional volume of fluid to the inflation device, and then perform a subsequent inflation of the balloon or expansion of the medical device using the additional fluid.

Another and/or alternative non-limiting object of the present disclosure is the provision of a method for accurately deploying a medical device comprising a) providing an inflation device that is configured to inflate a balloon and correspondingly expand a medical device or to at least partially expand a medical device; inserting or otherwise preparing the inflation device to include a certain amount of fluid in the inflation device to inflate the balloon or to expand a medical device; removably coupling a first inflation adjustment attachment device to the inflation device, and wherein having the first inflation adjustment attachment device has a predetermined thickness, and wherein the first inflation adjustment attachment device is configured to create a first stop on the inflation device to limit the amount of fluid the inflation device that can be inserted into the balloon or medical device, and wherein removal of the first inflation adjustment attachment device from the inflation device allows for a further incremental amount of fluid from the inflation device to be inserted into the balloon or medical device; and wherein the first inflation adjustment attachment device allows an administer to insert a first volume of fluid to the balloon or to the medical device until the first inflation adjustment attachment device prevents further insertion of fluid into the balloon or medical device; and wherein uncoupling of the first inflation adjustment attachment device from the inflation device enables an administer to add an additional volume of fluid to the balloon or medical device, wherein the additional volume of fluid corresponds to a predetermined thickness of the first inflation adjustment attachment device that was uncoupled from the inflation device.

Another and/or alternative non-limiting object of the present disclosure is the provision of a method for accurately deploying a medical device wherein a second inflation adjustment attachment device is removable coupled to the inflation device, and wherein the second inflation adjustment attachment device can have the same or different size and/or configuration form the first inflation adjustment attachment device, and wherein the second inflation adjustment attachment device also acts as another stop so as to limit the amount of fluid that the inflation device can insert into the balloon or medical device, and wherein the optional removal of the second inflation adjustment attachment device from the inflation device allows for a further incremental amount of fluid to be inserted from the inflation device into the balloon or medical device.

Another and/or alternative non-limiting object of the present disclosure is the provision of a method for deploying a medical device at a treatment area; the method comprising: a) providing an inflation device that is configured to inflate a balloon that correspondingly expands the medical device or to directly expand the medical device; b) preparing the inflation device with a predetermined amount of a material to inflate the balloon and/or to expand the medical device; c) removably coupling a first inflation adjustment attachment device to the inflation device, and wherein the first inflation adjustment attachment device is configured function as a first stop to limit an amount of the material inserted into the balloon or medical device during use of the inflation device; d) causing the inflation device to administer a first volume of the material to the balloon or the medical device until the first inflation adjustment attachment device prevents further material from being administered to the balloon or medical device; e) enabling the inflation device to allow further material to be inserted into the balloon that correspondingly further expands the medical device or to further directly expand the medical device by a) uncoupling the first inflation adjustment attachment device from the inflation device, and/or b) enabling a portion of the inflation device to move relative to the first inflation adjustment attachment device; and f) wherein a volume of the fluid that is further administered by the inflation device to the balloon or the medical device corresponds to a) the predetermined thickness of the first inflation adjustment attachment device that was previously coupled to the inflation device, and/or b) a distance that the portion of the inflation device moved relative to the first inflation adjustment attachment device.

Another and/or alternative non-limiting object of the present disclosure is the provision of a method for deploying a medical device at a treatment area wherein the step of enabling further comprises: I) uncoupling a second inflation adjustment attachment device from the inflation device, and/or II) enabling a portion of the inflation device to move relative to the second inflation adjustment attachment device; and wherein a volume of the fluid that is further administered by the inflation device to the balloon or the medical device corresponds to a) a predetermined thickness of the second inflation adjustment attachment device that was previously coupled to the inflation device, and/or b) a distance that the portion of the inflation device moved relative to the second inflation adjustment attachment device.

Another and/or alternative non-limiting object of the present disclosure is the provision of a method for deploying a medical device at a treatment area wherein a) the first inflation adjustment attachment device is uncoupled from the inflation device prior to the second inflation adjustment attachment device being uncoupled from the inflation device, or b) the second inflation adjustment attachment device is uncoupled from the inflation device prior to the first inflation adjustment attachment device being uncoupled from the inflation device.

Another and/or alternative non-limiting object of the present disclosure is the provision of a method for deploying a medical device at a treatment area wherein the predetermined thickness of the first inflation adjustment attachment device is the same or different from the predetermined thickness of the second inflation adjustment attachment device.

Another and/or alternative non-limiting object of the present disclosure is the provision of a method for deploying a medical device at a treatment area wherein the inflation device comprises: a) a housing that is configured to receive the material; and b) a plunger having a shaft, and wherein the plunger is at least partially movably disposed within the housing.

Another and/or alternative non-limiting object of the present disclosure is the provision of a method for deploying a medical device at a treatment area wherein the first inflation adjustment attachment device includes a body having a channel formed therein that is configured to receive at least a portion of the plunger.

Another and/or alternative non-limiting object of the present disclosure is the provision of a method for deploying a medical device at a treatment area wherein the first inflation adjustment attachment device is a clip, ring clip, C-clip, clasp, tab, or combinations thereof.

Another and/or alternative non-limiting object of the present disclosure is the provision of a method for deploying a medical device at a treatment area wherein the second inflation adjustment attachment device includes a body having a channel formed therein that is configured to receive at least a portion of the plunger.

Another and/or alternative non-limiting object of the present disclosure is the provision of a method for deploying a medical device at a treatment area wherein the second inflation adjustment attachment device is a clip, ring clip, C-clip, clasp, tab, or combinations thereof.

Another and/or alternative non-limiting object of the present disclosure is the provision of a method for deploying a medical device at a treatment area wherein the first inflation adjustment attachment device includes an interactive element in communication with a latch, and wherein activation of the interactive element causes the latch to disengage from a portion of the inflation device so that a portion of the inflation device can moved relative to the first inflation adjustment attachment device.

Another and/or alternative non-limiting object of the present disclosure is the provision of a method for deploying a medical device at a treatment area wherein the interactive element is a button, a knob, a switch, a lever, a grip, a controller, a toggle, or combinations thereof.

Another and/or alternative non-limiting object of the present disclosure is the provision of a method for deploying a medical device at a treatment area wherein the first inflation adjustment attachment device includes one or more audible, visual and/or haptic mechanisms that are configured to provide real-time feedback of an amount of the material delivered to the balloon or medical device.

Another and/or alternative non-limiting object of the present disclosure is the provision of a method for deploying a medical device at a treatment area wherein the first inflation adjustment attachment device includes identifying indicia, coloring or numbering that provides information about a volume of material that can be further inserted by the inflation device into the balloon or medical device after the first inflation adjustment attachment device is uncoupled from the inflation device.

Another and/or alternative non-limiting object of the present disclosure is the provision of a method for deploying a medical device at a treatment area wherein the material is a liquid or a gas.

Another and/or alternative non-limiting object of the present disclosure is the provision of a method for deploying a medical device at a treatment area wherein the medical device is a prosthetic valve, stent or angioplasty balloon.

Another and/or alternative non-limiting object of the present disclosure is the provision of an inflation adjustment attachment device for use with an inflation device; the inflation adjustment attachment device comprising: a) an arrangement for removably coupling the inflation adjustment attachment device to a plunger of an inflation device and wherein the inflation device is configured to inflate a balloon or medical device; and b) a body having predetermined thickness.

Another and/or alternative non-limiting object of the present disclosure is the provision of an inflation adjustment attachment device for use with an inflation device wherein the arrangement for removably coupling the inflation adjustment attachment device comprises a channel that is configured to temporarily retain the inflation adjustment attachment device on the plunger.

Another and/or alternative non-limiting object of the present disclosure is the provision of an inflation adjustment attachment device for use with an inflation device wherein the inflation adjustment attachment device is a clip, ring clip, C-clip, clasp, tab, or combinations thereof.

Another and/or alternative non-limiting object of the present disclosure is the provision of an inflation adjustment attachment device for use with an inflation device further including identifying indicia, coloring or numbering that provides information about a volume of material that can be further inserted by the inflation device into sad balloon or medical device after the first inflation adjustment attachment device is uncoupled from the inflation device.

These and other objects and advantages will become apparent to those skilled in the art upon reading and following the description taken together with the accompanying drawings.

### BRIEF DESCRIPTION OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. Reference may now be made to the drawings, which illustrate various embodiments that the disclosure may take in physical form and in certain parts and arrangement of parts wherein:

FIGS. 1A-1C depicts an exemplary, non-limiting inflation device/system having a non-limiting implementation of the disclosed inflation adjustment attachment device, wherein FIG. 1A depicts the inflation adjustment attachment device coupled to an inflation device filled with a predetermined volume of fluid/gas that may be used during a procedure, wherein FIG. 1B depicts administering a first amount of volume to a balloon until the inflation adjustment attachment device is engaged, and wherein FIG. 1C depicts the removal of the inflation adjustment attachment device and the administer of a retained amount of volume to the balloon;

FIG. 2 is a perspective view of the inflation adjustment attachment device of FIGS. 1A-1C, wherein the inflation adjustment attachment device has a first predetermined thickness;

FIG. 3 is a perspective view of the inflation adjustment attachment device of FIGS. 1A-1C, wherein the inflation adjustment attachment device has a second predetermined thickness, and wherein the second predetermined thickness is less than the first predetermined thickness of the inflation adjustment attachment device of FIG. 2;

FIGS. 4A and 4B depict another non-limiting implementation of the disclosed inflation adjustment attachment device that is configured for use with an inflation device/system, wherein the inflation adjustment attachment device is a C-clip;

FIG. 5 depicts a plurality of the inflation adjustment attachment device of FIGS. 4A and 4B coupled to a non-limiting inflation device/system;

FIG. 6 depicts still another non-limiting implementation of the disclosed inflation adjustment attachment device that is configured for use with an inflation system, wherein the inflation adjustment attachment device includes an interactive element coupled to a latch that, when activated, dispenses/administers an additional amount of volume contained in the inflation device; and

FIG. 7 depicts still another non-limiting implementation of the disclosed inflation adjustment attachment device that is configured for use with an inflation system, wherein the inflation adjustment attachment device is a pin.

### DESCRIPTION OF NON-LIMITING EMBODIMENTS

A more complete understanding of the articles/devices, processes and components disclosed herein can be obtained by reference to the accompanying drawings. These figures are merely schematic representations based on convenience and the case of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Percentages of elements should be assumed to be percent by weight of the stated element, unless expressly stated otherwise.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

For the sake of simplicity, the attached figures may not show the various ways (readily discernable, based on this disclosure, by one of ordinary skill in the art) in which the disclosed system, method and apparatus can be used in combination with other systems, methods and apparatuses. Additionally, the description sometimes uses terms such as “produce” and “provide” to describe the disclosed method. These terms are abstractions of the actual operations that can be performed. The actual operations that correspond to these terms can vary depending on the particular implementation and are, based on this disclosure, readily discernible by one of ordinary skill in the art.

Accurate deployment and placement of a prosthetic heart valve at a target site/area is an important part of the process of transcatheter aortic valve replacement (TAVR) and transcatheter aortic valve implantation (TAVI). Any additional time occupied during such a process/procedure could be detrimental to the proper function of the prosthetic heart valve and/or the life/health of the patient. Generally: (i) the prosthetic heart valves are crimped onto a deflated balloon of a balloon catheter or similar tube-like device; (ii) the balloon having the crimped valve is delivered to the predetermined treatment site/area within the patient; (iii) and the balloon is inflated via a fluid displacement device, syringe, or similar inflation device to correspondingly expand the prosthetic heart valve and lodge the prosthetic heart valve at the treatment site. During inflation of the balloon, additional balloon volume may be needed to achieve the necessary size/diameter of the prosthetic heart valve at the treatment site/area; however, current inflation devices allow for only one fill volume. As such, to add additional volume to the balloon and correspondingly increase the profile of the heart valve at the treatment site, current methodologies and devices require the user/physician deflate the balloon, add an additional amount of liquid to the inflation device, and then perform an additional post dilation of the valve with the additional amount of liquid. These additional steps increase the overall time of the procedure and increase the risk for vascular complications such as severe bleeding, sudden cardiac death, thromboembolic events, etc. Further, each inflation/deflation of the balloon increases the risk of thromboembolic events and damage to the leaflets and/or frame of the prosthetic heart valve.

Non-limiting implementations of the disclosed methods, systems and devices provide inflation adjustment attachment devices (e.g. clips, rings, ring clips, C-clips, latches, fasteners, tabs, clasps, pins, cotter pins, etc.) for inflation apparatuses (e.g. endoflators, fluid and/or gas displacement/delivery devices, syringes, etc.) and methods of using the same that enable a predetermined amount of fluid/gas volume within the inflation apparatus to be used if additional inflation volume of the balloon and a larger size/diameter of the prosthetic heart valve is warranted. In one non-limiting embodiment, the disclosed inflation adjustment attachment devices and methods allow a user (e.g. physician, surgeon, nurse, etc.) to incrementally increase inflation volume of a balloon for a balloon inflatable device (e.g., balloon expandable prosthetic valve, balloon expanded stent, etc.) or for an angioplasty balloon, without having to deflate the balloon, add additional volume to the inflation device, and perform post dilation of the balloon with the additional volume. Although the present disclosure will particularly discuss inflation adjustment attachment devices and methods for use with prosthetic valves (e.g., prosthetic heart valves), it will be appreciated that the inflation adjustment attachment devices and methods in accordance with the present disclosure can be used with other types of expandable medical devices (e.g., stent, angioplasty balloon, catheters, etc.) that are expanded within a patient at a predetermined treatment area.

In one or more exemplary, non-limiting embodiments, a plurality of any of the disclosed inflation adjustment attachment devices (of the same and/or varying thickness) can be used with the inflation apparatus to achieve the predetermined amount of fluid/gas volume to be retained within the inflation apparatus for use if additional inflation volume of the balloon and a larger size/diameter of the prosthetic heart valve is warranted. In one or more exemplary, non-limiting embodiments, the disclosed inflation adjustment attachment devices include identifying indicia (e.g., engraved, labels, print, number, etc.) and/or be color coded according to the volume of injection solution associated with the thickness of the inflation adjustment attachment devices.

In one non-limiting embodiment, the thickness of the disclosed inflation adjustment attachment devices can be adjusted/fabricated to correspond to a retained volume range of at least 0.01 mL (e.g., 0.01 mL to 20 mL and all values and ranges therebetween).

In one or more exemplary, non-limiting embodiments, any of the disclosed inflation adjustment attachment devices can advantageously include one or more audible and/or haptic mechanisms that are configured to provide feedback when: (i) the inflation target of the balloon is achieved; (ii) the size/diameter target of prosthetic heart valve is achieved; (iii) the balloon volume is less than the inflation target; (iv) the prosthetic heart valve size/diameter is less than the target; (v) the balloon volume is great than the inflation target; and/or (vi) the prosthetic heart valve size/diameter is greater than the target. These one or more audible and/or haptic mechanisms can provide real-time feedback of the amount of volume presently delivered to the balloon, thereby reducing procedure time (e.g., the user does not have to read the inflation device to determine the amount of volume dispensed) and/or mitigating the risk of under- and/or over-expansion of the prosthetic valve.

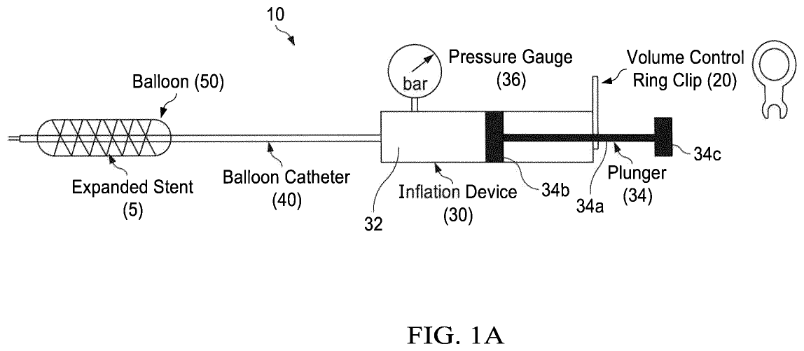

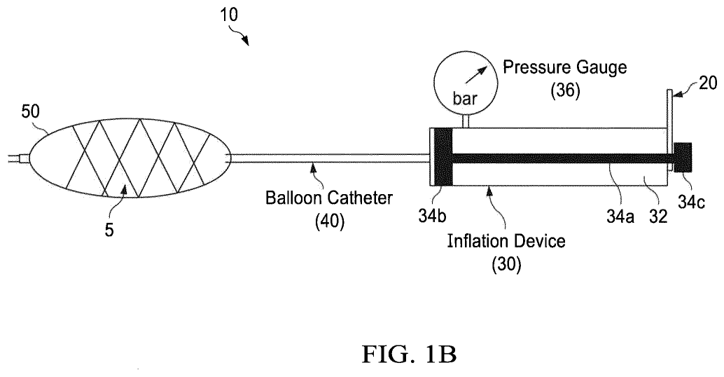

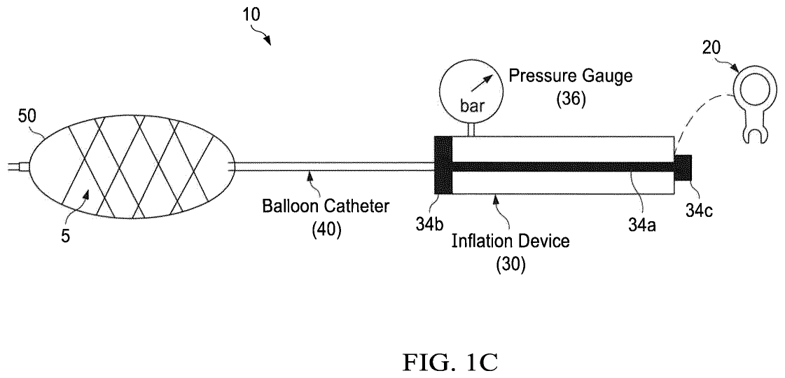

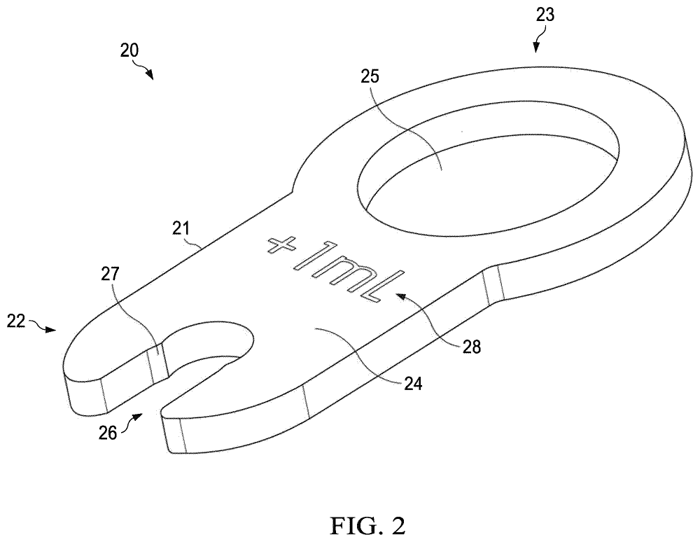

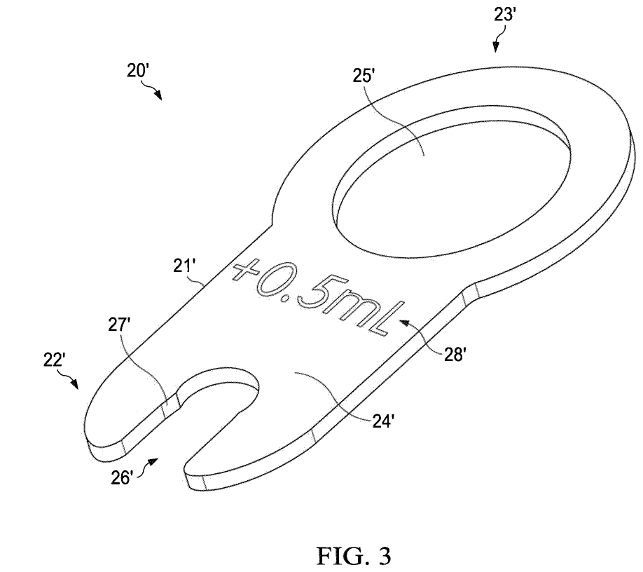

FIGS. 1A-1C depict an exemplary, non-limiting inflation device/system having a non-limiting implementation of the disclosed inflation adjustment attachment device, wherein FIG. 1A depicts the inflation adjustment attachment device 20 coupled to an inflation device 30 filled with a predetermined volume of fluid/gas that may be used during a procedure. FIG. 1B depicts administering a first amount of volume to a balloon until the inflation adjustment attachment device 20 is engaged. FIG. 1C depicts the removal of the inflation adjustment attachment device 20 and the administration of a retained amount of volume in the inflation device 30 to the balloon 50. FIG. 2 is a perspective view of the non-limiting inflation adjustment attachment device 20 of FIGS. 1A-1C, wherein the inflation adjustment attachment device has a first predetermined thickness. FIG. 3 is a perspective view of another inflation adjustment attachment device, wherein the inflation adjustment attachment device has a second predetermined thickness, and wherein the second predetermined thickness is less than the first predetermined thickness of the inflation adjustment attachment device of FIG. 2.

Referring again to FIGS. 1A-1C, there is disclosed is an exemplary, non-limiting inflation system 10 that is configured to expand a medical device 5 (e.g., prosthetic valve, TAV, stent, balloon catheter, angioplasty balloon, etc.) at a predetermined target area/site or treatment area within a patient. The inflation system 10 includes an inflation adjustment attachment device 20; an inflation device 30 having a housing/chamber 32, a plunger/piston 34 that is at least partially disposed within the housing/chamber 32, and an optional pressure gauge 36; a catheter or similar tube-like device 40 that is coupled to the inflation device 30; and a balloon 50 formed on and/or in fluid connection with the catheter 40. Although FIG. 1A illustrates a medical device 5 positioned on balloon 50, it can be appreciated that balloon 50 itself can be the medical device such as in procedures as balloon angioplasty procedures, and the like. FIG. 1A is merely illustrative of a non-limiting configuration for inserting a fluid into balloon 50 or some other inflatable medical device at or near a distal region of the catheter 40. It can be appreciated that many other configurations of the inflation system 10 can be used that incorporates the use of one or more inflation adjustment attachment devices 20.

When the medical device 5 is a crimpable medical device (e.g., stent, heart valve, TAV, etc.), the medical device 5 is typically crimped or collapsed onto at least a portion of balloon 50.

The plunger/piston 34 includes a shaft 34a having a base portion 34b at the distal end or end region of the shaft 34a, and an optional handle 34c. As can be appreciated, many configurations of the plunger/piston 34 can be used.

In the present non-limiting embodiment, the inflation adjustment attachment device 20 is removably coupled to shaft 34a of the plunger/piston 34 at a location that is prior proximal end of housing/chamber 32. In other non-limiting, exemplary embodiments, the inflation adjustment attachment device 20 is coupled (e.g., removably coupled, etc.) to the shaft 34a at any location between the base portion 34b and the handle 34c. In other non-limiting, exemplary embodiments, the inflation adjustment attachment device 20 is coupled (e.g., removably coupled, etc.) to housing/chamber 32 at any predetermined location thereon.

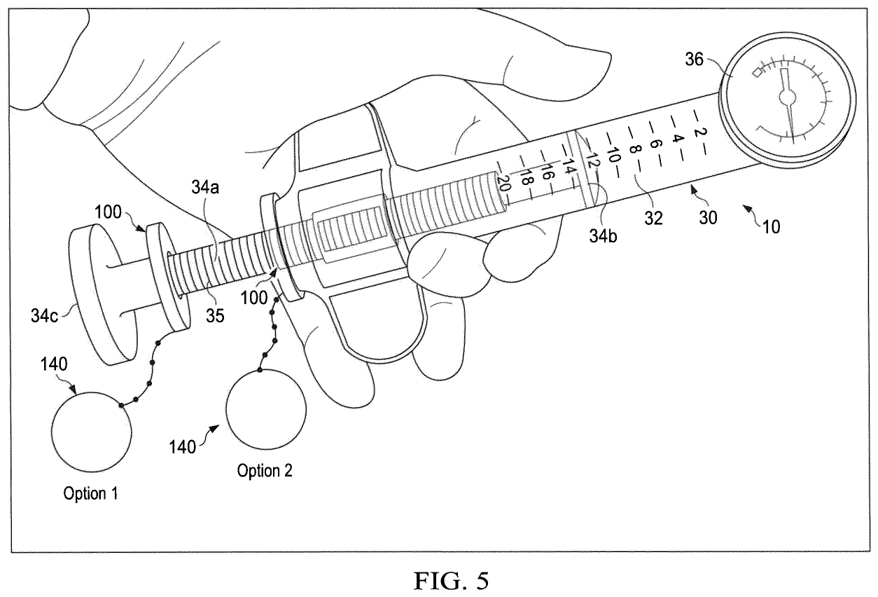

In one non-limiting embodiment, the housing/chamber 32 optionally includes identifying volumetric/measurement indicia to determine the volume of fluid contained therein (see for example FIGS. 5-7). In one non-limiting embodiment, the shaft 34a of the plunger/piston 34 is threaded and the handle 34c is rotated to linearly advance the plunger/piston 34 within the housing/chamber 32. In another one non-limiting embodiment, shaft 34a of the plunger/piston 34 is non-threaded and a force is applied to the handle 34c to linearly advance the plunger/piston 34 within the housing/chamber 32. Although not shown, it is to be appreciated that the inflation system 10 further includes a dispenser, hose or similar tube-like structure that allows the housing/chamber 32 to receive an amount (e.g., predetermined amount, etc.) of fluid/gas.

Now referring to FIGS. 1A, 1B, 1C, 2 and 3, one non-limiting configuration of inflation adjustment attachment device is illustrated. The inflation adjustment attachment device 20, 20′ includes a body 21, 21′ having a first end 22, 22′, a second end 23, 23′, a first surface 24, 24′, and a second surface (not shown) that is opposite the first surface 24, 241. An opening 25 is optionally formed in the body 21, 21′ near the second end 23, 23′. A channel 26, 26′ is optionally formed in the first end 22, 22′. The channel 26, 26′ is configured to receive a portion of the shaft 34a of the plunger/piston 34. In the present non-limiting embodiment, the channel 26, 26′ optionally includes one or more stops 27, 27′ (e.g., detents, indents, releases, etc.) that are configured to releasably retain/secure the inflation adjustment attachment device 20 to shaft 34a. As illustrated in FIGS. 2 and 3, the stops 27, 27′ extend inwardly into the channel 26, 26′ to make the channel narrower at the location of the stops 27, 27′. The stops 27, 27′ optionally have a rounded surface to facilitate in the stops 27, 27′ moving over the shaft 34a when inserting/removing the inflation adjustment attachment device 20, 20′ on/from the shaft 34a. The shape and size of the channel 26/26′ that is located above the stops 27, 27′ is such that at least 51% (e.g., 51-100% and all values and ranges therebetween) of the shaft is located above the stops 27, 27′ when the inflation adjustment attachment device 20, 20′ is positioned on the shaft 34a. As can be appreciated, other or additional arrangement can be used to releasably retain/secure the inflation adjustment attachment device 20 to shaft 34a.

In one non-limiting embodiment, the optional opening 25, 25′ can be configured as a grip for the user when handling the inflation adjustment attachment device 20, 21′ for securing and/or removing the inflation adjustment attachment device 20, 21′ from/to the inflation device 30.

In one non-limiting embodiment, the inflation adjustment attachment device 20, 21′ is a clip. In one non-liming embodiment, the inflation adjustment attachment device 20, 21′ is a ring-clip. In one non-liming embodiment, the inflation adjustment attachment device 20, 21′ is a fastener. In one non-liming embodiment, the inflation adjustment attachment device 20, 21′ is a clasp. In one non-liming embodiment, the inflation adjustment attachment device 20, 21′ is a tab.

Still referring to FIGS. 1A, 1B, 1C, 2 and 3, in the present non-limiting embodiment, the inflation adjustment attachment device 20, 20′ has a first predetermined thickness about channel 26, 26′ that substantially corresponds a particular volume of fluid to be retained within the inflation device 30 after the inflation adjustment attachment device 20, 20′ prevents further advancement of the plunger 34 in the housing/chamber 32, thereby preventing further dispensement of fluid from the inflation device by the inflation adjustment attachment device 20. The thickness of the inflation adjustment attachment device 20, 20′ has a direct relationship to the amount of volume to be retained within the inflation device 30 and to be dispensed from the inflation device after the inflation adjustment attachment device 20, 20′ is removed from the inflation device. In one non-limiting embodiment, the inflation adjustment attachment device 20, 20′ optionally includes identifying indicia 28, 28′ disposed on the body 21, 21′ on the first surface 25, 25′ and/or the second surface. The identifying indicia 28, 28′ can include, in some non-limiting embodiments, a volume retention indication in the housing/chamber 32 that substantially corresponds to the thickness of the inflation adjustment attachment device 20, 201. In the present non-limiting embodiment, the first predetermined thickness of the inflation adjustment attachment device 20 corresponds to 1 mL of volume to be retained within the housing/chamber 32 inflation device 30 after the inflation adjustment attachment device 20 prevents further advancement of the plunger 34 in the housing/chamber 32. As such, after the plunger 34 is prevented from further dispensing fluid from the inflation device 30 by the inflation adjustment attachment device 20, the removal of the inflation adjustment attachment device 20 from the inflation device 30 will allow the plunger 34 to further move into the housing/chamber 32 of the inflation device 30 and dispense up to 1 mL of additional fluid from the inflation device 30. The amount of additional fluid dispensed from the inflation device 30 will correspond to the distance of movement of the plunger 34, which distance corresponds to the thickness of the removed inflation adjustment attachment device 20. As illustrated in FIG. 3, the first predetermined thickness of the inflation adjustment attachment device 20′ corresponds to 0.5 mL of volume to be retained within the housing/chamber 32 of the inflation device 30 after the inflation adjustment attachment device 20 prevents further advancement of the plunger 34 in the housing/chamber 32. As such, after the plunger 34 is prevented from further dispensing fluid from the inflation device 30 by the inflation adjustment attachment device 20, the removal of the inflation adjustment attachment device 20 from the inflation device 30 will allow the plunger 34 to further move into the housing/chamber 32 of the inflation device 30 and dispense up to 0.5 mL of additional fluid from the inflation device 30. The amount of additional fluid dispensed from the inflation device 30 will correspond to the distance of movement of the plunger 34, which distance corresponds to the thickness of the removed inflation adjustment attachment device 20. As can be appreciated, when the inflation adjustment attachment device prevents further movement of the shaft into the chamber, the inflation adjustment attachment device can also or alternatively indicate the amount of fluid that has been inserted into the balloon.

As can be appreciated, multiple inflation adjustment attachment device can be simultaneously connected to the plunger 34. For example, both inflation adjustment attachment devices 20 and 20′ could be simultaneously connected to the plunger 34. As such, when the plunger 34 is prevented from further advancing in the housing/chamber 32 of the inflation device 30, 1.5 mL of fluid will remain in within the housing/chamber 32 of the inflation device 30. Thereafter, the user can determine if additional fluid needs to be inserted into the balloon 50. Such determination can be made by any number of techniques (e.g., X-ray, ultrasound, other types of imaging, volume inflation detector for balloon, etc.). If additional inflation of the balloon is required, the user can then determine the amount of additional fluid required. For instance, if only 0.5 mL of fluid is required, the 0.5 ml inflation adjustment attachment device 20′ can be removed from the shaft 34a while the inflation adjustment attachment device 20 remains on shaft 34a, and the shaft 34a is then advanced after removal of inflation adjustment attachment device 20′ to insert 0.5 ml of fluid into the balloon 50. However, if only 1 mL of fluid is required, the 1 ml inflation adjustment attachment device 20 can be removed from the shaft 34a while the inflation adjustment attachment device 20′ remains on shaft 34a, and the shaft 34a is then advanced after removal of inflation adjustment attachment device 20 to insert 1 ml of fluid into the balloon 50. Furthermore, if 1.5 mL of fluid is required, both inflation adjustment attachment device 20, 20′ can be removed from the shaft 34a, and the shaft 34a is then advanced after removal of inflation adjustment attachment devices 20, 20′ to insert 1.5 ml of fluid into the balloon 50. As can be appreciated, any combination of inflation adjustment attachment devices that have the same or different thicknesses can be removably connected to the shaft 34a to allow the user to insert the needed amount of fluid into the balloon 50. As can be appreciated, the number of inflation adjustment attachment devices connected to the shaft 34a is non-limiting (e.g., 1-20 inflation adjustment attachment devices connected to the shaft 34a and all values and ranges therebetween). As can be appreciated, when two of more inflation adjustment attachment devices are connected to the shaft 34a, the additional fluid volume allowed by two or more of the inflation adjustment attachment devices when removed from the shaft 34a can be the same or different.

In some non-limiting embodiments, the inflation adjustment attachment device 20, 21′ is color coded according to the volume of fluid that is further dispensable from the removal of the inflation adjustment attachment device 20, 20′ and the thickness of the inflation adjustment attachment device 20, 20′. It is to be appreciated that the thickness of the inflation adjustment attachment device 20, 20′ can be adjusted to obtain certain amounts of additional fluid dispensing from the inflation device 30 when the inflation adjustment attachment device 20, 20′ is removed from the inflation device (e.g., 0.01 mL, 0.1 mL, 0.5 mL, 1 mL etc.).

Still referring to FIGS. 1A, 1B, 1C, 2 and 3, in accordance with one non-limiting aspect of the present disclosure: (i) the inflation device 30 is filled with a maximum (e.g. an upper limit) inflation volume of fluid/gas the user may need use during a procedure, which maximum inflation, as determined from pre-procedure planning (e.g. imaging, electrocardiogram testing, etc.) and patient diagnostic information; (ii) one or more the inflation adjustment attachment devices 20, 20′ are fastened/attached to the shaft 34a of the plunger/piston 34 at a location thereon before the housing/chamber 32; (iii) the plunger/piston 34 is advanced until one of the inflation adjustment attachment devices 20, 20′ prevents further advancement of the plunger/piston 34, thereby resulting in the administering a first amount of volume to the balloon 50; and (iv) if an additional amount of volume in the balloon 50 (i.e. additional size/diameter of the prosthetic valve) is warranted, one or more of the inflation adjustment attachment devices 20, 20′ is removed, and the plunger/piston 34 is then advanced to administer further fluid to the balloon 50 as allowed by the removal of the inflation adjustment attachment device 20.

Still referring to FIGS. 1A, 1B, 1C, 2 and 3, in accordance with another non-limiting aspect of the present disclosure: (i) the inflation device 30 is filled with a maximum (e.g. an upper limit) inflation volume of fluid/gas the user may use during the procedure, as determined from pre-procedure planning (e.g. imaging, electrocardiogram testing, etc.) and patient diagnostic information; (ii) a plurality of inflation adjustment attachment devices 20, 20′ are fastened/attached to the shaft 34a of the plunger/piston 34 at predetermined locations thereon before the housing/chamber 32 (e.g., the plurality of inflation adjustment attachment devices 20 are positioned adjacent to one another, one or more of the plurality of inflation adjustment attachment devices 20 are spaced from one another, etc.); (iii) the plunger/piston 34 is advanced until one of the inflation adjustment attachment devices 20, 20′ prevents further advancement of the plunger/piston 34, thereby resulting in the administering a first amount of volume to the balloon 50; and (iv) if an additional amount of volume in the balloon 50 is warranted (i.e. additional size/diameter of the prosthetic valve), one or more of the plurality of inflation adjustment attachment devices 20, 20′ is removed, and the plunger/piston 34 is then advanced to administer further fluid to the balloon 50 as allowed by the removal of the inflation adjustment attachment device 20. (administering a first retained amount of volume to the balloon 50); and (v) if still an additional amount of volume in the balloon 50 is warranted and one or more of the inflation adjustment attachment devices 20, 20′ are still removably connected to the plunger, one or more additional inflation adjustment attachment devices 20 can be removed and the plunger/piston 34 to allow the plunger to further advance in the inflation device and supply additional fluid to the balloon 50. It is to be appreciated that a plurality of inflation adjustment attachment devices 20, 20′ can be used with the inflation device 30.

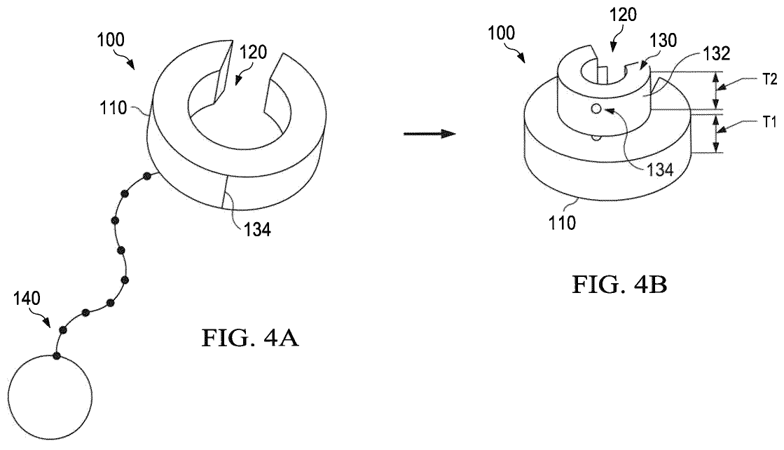

Referring now to FIGS. 4A and 4B, FIGS. 4A and 4B depict another non-limiting implementation of the disclosed inflation adjustment attachment device 100 that is configured for use with an inflation device/system, wherein the inflation adjustment attachment device 100 is a C-clip. FIG. 5 depicts a plurality of the inflation adjustment attachment devices 100 of FIGS. 4A and 4B coupled to a non-limiting inflation device/system. The inflation adjustment attachment devices 100 illustrated in FIGS. 4A, 4B and 5 have a ring 110 and tether arrangement 140 to facilitate in the removal of the inflation adjustment attachment device 110 from a plunger.

Now referring to FIGS. 1A-1C, 4A and 4B, there is provided is an exemplary, non-limiting inflation adjustment attachment device 100 that is configured for use with the exemplary, non-limiting inflation system 10 previously discussed and illustrated in FIG. 1A-1C. In the present non-limiting embodiment, the inflation adjustment attachment device 100 is used in lieu of the inflation adjustment attachment devices 20 and/or 20′; however, it is to be appreciated that the inflation adjustment attachment device 100 could be used in combination with any of the inflation adjustment attachment devices disclosed herein. In the present non-limiting embodiment, the inflation adjustment attachment device 100 includes a body 110 having a channel 120 formed therein that is configured to receive and secure (e.g., removably secure, etc.) to the shaft 34a of the plunger/piston 34. The inflation adjustment attachment device 100 optionally includes a chain and ring arrangement to facilitate in the removal of the inflation adjustment attachment device 100 from the shaft 34a. As can be appreciated, other or additional arrangements can be to facilitate in the removal of the inflation adjustment attachment device 100 from the shaft 34a.

The inflation adjustment attachment device 100 optionally includes one or more audible and/or haptic mechanisms 130 (e.g. ball detent, etc.) to provide feedback (e.g., audible feedback, tactile feedback, etc.) to the user when: (i) the inflation target of the balloon is achieved; (ii) the size/diameter target of medical device (e.g., balloon, prosthetic heart valve, etc.) is achieved; (iii) the balloon volume is less than the inflation target; (iv) the medical device (e.g., balloon, prosthetic heart valve, etc.) size/diameter is less than the target; (v) the balloon volume is great than the inflation target; and/or (vi) the (e.g., balloon, prosthetic heart valve, etc.) size/diameter is greater than the target.

As best illustrated in FIG. 4B, the haptic mechanism 130, when used, can include a raised flange 132 and rib, ball or rod 134 (e.g., biased ball, biased ball, etc.). The shaft 34a of the plunger/piston 34 can optionally include ribs 35, indents, etc. such that when the shaft 34a moves past the haptic mechanism 130, a sound and/or tactile indication is heard and/or felt by the user to make the user aware of the progress of movement of the plunger/piston 34 during the inflation of the balloon. The sound and/or the resistance past the haptic mechanism 130 as the plunger/piston 34 moves past the haptic mechanism 130 can be the same, or the resistance can change and/or the sound can change as the plunger/piston 34 is moved further into the housing/chamber 32. In one non-limiting arrangement, the inflation adjustment attachment device 100 abuts against the proximal end of the housing/chamber 32, and the plunger/piston 34 is moved into the housing/chamber 32 while the inflation adjustment attachment device 100 remains essentially stationary against the housing/chamber 32.

As can be appreciated, the haptic mechanism 130 can alternatively or additionally be used to connect to a portion of the shaft 34a of the plunger/piston 34, wherein the inflation adjustment attachment device 100 is spaced from the proximal end of the housing/chamber 32 prior to the plunger/piston 34 being moved into the housing/chamber 32 to cause inflation of the balloon 50. In such an arrangement, the inflation adjustment attachment device 100 moves with the shaft 34a as the plunger/piston 34 being moved into the housing/chamber 32. When the inflation adjustment attachment device 100 contacts the proximal end of the housing/chamber 32, the user understands that a certain volume of fluid has been inserted into the balloon. If the plunger 34c or end region of the shaft 34a is still spaced from the proximal end of the inflation adjustment attachment device 100, the shaft 34a can be optionally further moved into the housing/chamber 32 and the haptic mechanism 130 can optionally provide audible and/or tactile indication for the amount of additional fluid being added to the balloon (e.g., each audible and/or tactile indication can represent that a certain amount of fluid was added to the balloon). The spacing of the ribs, notches, etc. on the shaft 34a that cause the audible and/or tactile indication by the haptic mechanism 130 can be selected to correspond to a certain amount of fluid being added to the balloon. Alternatively or additionally, the inflation adjustment attachment device 100 can be removed from the shaft 34a to enable the shaft 34a to further move into the housing/chamber 32 to further add fluid to the balloon.

As can be appreciated, a) the inflation adjustment attachment device 100 can be configured to slide on an outer surface of the shaft 34a, b) the shaft 34a can be configured to slide past the inflation adjustment attachment device 100, c) the inflation adjustment attachment device 100 can be configured to threadedly move on threaded outer surface of the shaft 34a by rotating the shafted and/or the inflation adjustment attachment device 100, or d) the shaft 34a can be configured to threadedly move past the inflation adjustment attachment device 100 by rotating the shafted and/or the inflation adjustment attachment device 100. As can be appreciated, other arrangements can be used for allowing movement of the shaft relative to the inflation adjustment attachment device 100.

The desired thickness of the inflation adjustment attachment device 100 can be obtained in multiple ways. As illustrated in FIGS. 2 and 3, the body of the inflation adjustment attachment device has a generally uniform thickness. As illustrated in FIG. 4B, the thickness of the inflation adjustment attachment device is a combination of the body thickness T1 and the thickness of the raised flange T2. Thicknesses T1 and T2 can be the same or different.

As with the inflation adjustment attachment devices 20 and 20′, the inflation adjustment attachment device 100 includes a predetermined thickness that substantially corresponds to the amount of volume to be retained within the inflation device 30. The thickness of the inflation adjustment attachment device 100 has a direct relationship to the amount of volume to be retained within the inflation device 30 when the inflation adjustment attachment device 100 is connected to shaft 34a. As can be appreciated, when the inflation adjustment attachment device prevents further movement of the shaft into the chamber, the inflation adjustment attachment device can also or alternatively indicate the amount of fluid that has been inserted into the balloon.

In some non-limiting embodiments, the inflation adjustment attachment device 100 includes identifying indicia disposed on the body 110. The identifying indicia can include, in some non-limiting embodiments, a volume retention indication that substantially corresponds to the thickness of the inflation adjustment attachment device 100. In the present non-limiting embodiment, the predetermined thickness of the inflation adjustment attachment device 100 corresponds to 1 mL of volume to be retained within the inflation device 30; however, other corresponding volumes can be used. In some non-limiting embodiments, the inflation adjustment attachment device 100 is color coded according to the volume of injection solution associated with the thickness of the inflation adjustment attachment device 100. It is to be appreciated that the thickness of the inflation adjustment attachment device 100 can be selected depending on the amount of volume to be retained, for example +0.5 mL, +1 mL, etc. In one non-limiting embodiment, thickness of the inflation adjustment attachment device 100 can be adjusted/fabricated to correspond to a retained volume range of at least 0.01 ml (e.g., 0.01 ml to 20 mL and all values and ranges therebetween).

Still referring to FIGS. 1A-1C, 4A and 4B, in accordance with one non-limiting aspect of the present disclosure: (i) the inflation device 30 is filled with a maximum (e.g. an upper limit) inflation volume of fluid/gas the user may use during the procedure, as determined from pre-procedure planning (e.g. imaging, electrocardiogram testing, etc.) and patient diagnostic information; (ii) the inflation adjustment attachment device 100 is fastened/attached to the shaft 34a of the plunger/piston 34 at a location thereon before the housing/chamber 32; (iii) the plunger/piston 34 is advanced until the inflation adjustment attachment device 100 prevent further advancement of the plunger/piston 34 in the housing/chamber 32, thereby administering a first amount of volume to the balloon 50 and providing a feedback response to the user that a first amount of volume has been administered; and (iv) if an additional amount of volume in the balloon 50 (i.e. additional size/diameter of the prosthetic valve) is warranted, the inflation adjustment attachment device 100 can be removed from the shaft 34a, and the plunger/piston 34 can thereafter be advanced even further to administer an additional volume into the balloon 50.

In one non-limiting embodiment, a plurality of inflation adjustment attachment devices 100 are used with the inflation system 10 (see FIG. 5). In one non-limiting embodiment, one or more inflation adjustment attachment devices 100 having a first predetermined thickness (corresponding to a first volume) are used with one or more inflation adjustment attachment devices 100 having a second predetermined thickness (corresponding to a second volume).

Now referring to FIGS. 4A, 4B and 5, in accordance with another non-limiting aspect of the present disclosure: (i) the inflation device 30 is filled with a maximum (e.g. an upper limit) inflation volume of fluid/gas the user may use during the procedure, as determined from pre-procedure planning (e.g. imaging, electrocardiogram testing, etc.) and patient diagnostic information; (ii) a plurality of inflation adjustment attachment devices 100 are fastened/attached to the shaft 34a of the plunger/piston 34 at predetermined locations thereon before the housing/chamber 32; (iii) the plunger/piston 34 is advanced until the plurality of inflation adjustment attachment devices 100 prevent further advancement of the plunger/piston 34, thereby administering a first amount of volume to the balloon 50; and (iv) if an additional amount of volume in the balloon 50 is warranted (i.e. additional size/diameter of the prosthetic valve), one of the plurality of inflation adjustment attachment devices 100 can be removed, and the plunger/piston 34 can be advanced until the remaining inflation adjustment attachment devices 100 in the shaft 34a prevent further advancement of the plunger/piston 34 thereby administering a first retained amount of volume to the balloon 50; and (v) if still an additional amount of volume in the balloon 50 is warranted, the another one of the plurality of inflation adjustment attachment devices 100 can be removed to allow for further advancement of the shaft 34a. It will be appreciated that any of a variety of additional or alternative steps may be included in a method of using the inflation adjustment attachment device 100.

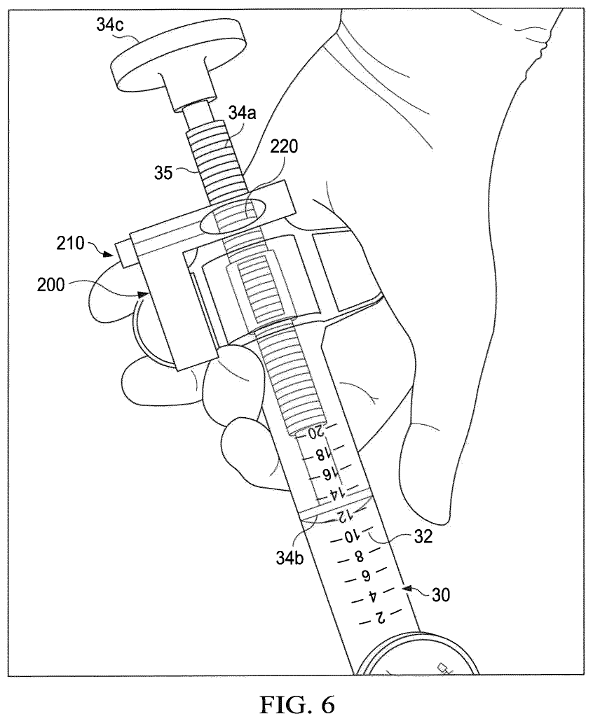

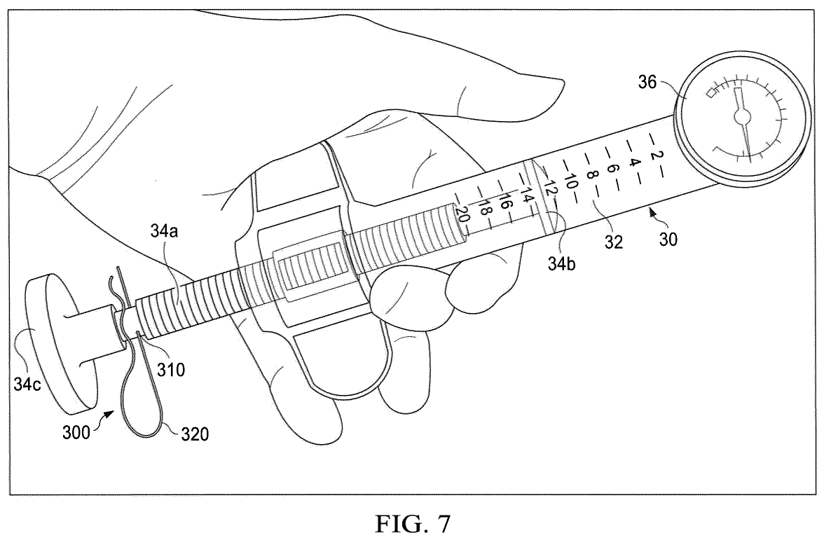

FIG. 6 depicts still another non-limiting implementation of the disclosed inflation adjustment attachment device 200 that is configured for use with an inflation system, wherein the inflation adjustment attachment device 200 includes an interactive element coupled to a latch 220 that, when activated, allows further movement of the shaft 34a relative to the inflation adjustment attachment device 200 to allow for additional, retained amount of fluid in the housing/chamber 32 to be inserted into the ballon 50.

Referring now to FIGS. 1A-IC and 6, there is provided an exemplary, non-limiting inflation adjustment attachment device 200 that is configured for use with the exemplary, non-limiting inflation system 10 previously discussed and illustrated in FIG. 1A-IC. In the present non-limiting embodiment, the inflation adjustment attachment device 200 is used in lieu of the forgoing inflation adjustment attachment devices disclosed herein (e.g. 20, 20′, 100); however, it is to be appreciated that the inflation adjustment attachment device 200 could be used in combination with any of the inflation adjustment attachment devices disclosed herein. In the present non-limiting embodiment, the inflation adjustment attachment device 200 includes an interactive element 210 (e.g., button, spring biased button, knob, switch, lever, handle, controller, toggle, touch sensor, etc.) in mechanical and/or electrical communication with a latch 220. When the interactive element 210 is activated, the latch 220 correspondingly moves (i.e. disengages from the shaft 43a; disengages from the ribs 35, indents, etc. on the outer surface of the shaft 34a) and allows the piston/plunger 34 to advance past the inflation adjustment attachment device 200 and then further within the housing/chamber 32 so that a retained amount of fluid in the housing/chamber 32 can be inserted into the ballon 50.

The inflation adjustment attachment device 200 can a) includes a predetermined thickness that substantially corresponds the amount of volume to be retained within the inflation device 30, and/or b) the inflation adjustment attachment device 200 can be positioned on the shaft 34a in a certain location to indicate the amount of fluid that has been inserted into the balloon when the inflation adjustment attachment device 200 prevents further movement of the shaft 34a into the chamber 32. The thickness of the inflation adjustment attachment device 200 has a direct relationship to the amount of volume to be retained within the inflation device 30. In the present non-limiting embodiment, activating the interactive element 210 allows an additional volume to be administered to the balloon 50. In one non-limiting embodiment, the thickness of the inflation adjustment attachment device 200 can be adjusted/fabricated to correspond to a retained volume range. As can be appreciated, when the inflation adjustment attachment device prevents further movement of the shaft into the chamber, the inflation adjustment attachment device can also or alternatively indicate the amount of fluid that has been inserted into the balloon.

In some non-limiting embodiments, the inflation adjustment attachment device 200 includes identifying indicia disposed thereon. The identifying indicia can include, in some non-limiting embodiments, a volume retention indication that substantially corresponds to the thickness of the inflation adjustment attachment device 200. In some non-limiting embodiments, the inflation adjustment attachment device 200 is color coded according to the volume of injection solution associated with the thickness of the inflation adjustment attachment device 200.

Still referring to FIGS. 1A-1C and 6, in accordance with one non-limiting aspect of the present disclosure: (i) the inflation device 30 is filled with a maximum (e.g. an upper limit) inflation volume of fluid/gas the user may use during the procedure, as determined from pre-procedure planning (e.g. imaging, electrocardiogram testing, etc.) and patient diagnostic information; (ii) one or more inflation adjustment attachment devices 200 are fastened/attached at least partially to the shaft 34a of the plunger/piston 34 at a location proximal to the front end of the housing/chamber 32; (iii) the plunger/piston 34 is advanced until the handle 34c engages the one or more inflation adjustment attachment devices 200, thereby administering a first amount of volume to the balloon 50; and (iv) if an additional amount of volume in the balloon 50 (i.e. additional size/diameter of the prosthetic valve) is warranted, the latch 220 is moved/opened by way of the interactive element 210 on the one or more inflation adjustment attachment devices 200, and the plunger/piston 34 can be advanced even further to administer the retained amount of volume to the balloon 50.

Referring to FIGS. 5 and 6, the inflation adjustment attachment device 100, 200 can optionally be threaded on a portion of the shaft of the plunger. As such, the inflation adjustment attachment device 100, 200 can be adjustably positioned along the longitudinal length of the shaft of the plunger by rotating the inflation adjustment attachment device 100, 200 on the shaft. As such, additional amounts of fluid dispensing from the inflation device can be accomplished by the repositioning of the inflation adjustment attachment device 100, 200 along the longitudinal length of the shaft. As can be appreciated, other arrangement can be used to enable the adjustment of the inflation adjustment attachment device 100, 200 along the longitudinal length of the shaft.

It will be appreciated that any of a variety of additional or alternative steps may be included in a method of using the inflation adjustment attachment device 200.

In one non-limiting embodiment, a plurality of inflation adjustment attachment devices 200 are used with the inflation system 10. In one non-limiting embodiment, one or more inflation adjustment attachment devices 200 having a first predetermined thickness (corresponding to a first volume) are used with one or more inflation adjustment attachment devices 200 having a second predetermined thickness (corresponding to a second volume).

Referring now FIG. 7, FIG. 7 depicts still another non-limiting implementation of the disclosed inflation adjustment attachment device 300 that is configured for use with an inflation system, wherein the inflation adjustment attachment device 300 is or includes a pin 320.

Now referring to FIGS. 1A-1C and 7, provided is an exemplary, non-limiting inflation adjustment attachment device 300 that is configured for use with the exemplary, non-limiting inflation system 10 previously discussed and illustrated in FIG. 1A-1C. In the present non-limiting embodiment, the inflation adjustment attachment device 300 is used in lieu of the forgoing inflation adjustment attachment devices disclosed herein (e.g. 20, 20′, 100, 200); however, it is to be appreciated that the inflation adjustment attachment device 300 could be used in combination with any of the inflation adjustment attachment devices disclosed herein. In the present non-limiting embodiment, the inflation adjustment attachment device 300 is configured to engage a hole or aperture 310 formed at a predetermined location along the shaft 34a of the plunger/piston 34. It is to be appreciated that the hole or aperture 310 can be formed at any location (or more than one location) along the shaft 34a. It is to be appreciated that the hole or aperture 310 can be formed on any like part of the inflation device 30 (e.g., the handle 34c, etc.)

In one non-limiting embodiment, the inflation adjustment attachment device 300 is or includes a pin 320 that couples with the inflation device 30. In one non-liming embodiment, the pin 320 of the inflation adjustment attachment device 300 is a cotter pin that couples with the inflation device 30.

The location that the inflation adjustment attachment device 300 is connected to the shaft 34a corresponds to the amount of volume to be retained within the inflation device 30 after the shaft is moved in the chamber housing/chamber 32 and the inflation adjustment attachment device 300 has engaged the chamber housing/chamber 32 to prevent further movement of the shaft 34a into the chamber housing/chamber 32. The connection location of the inflation adjustment attachment device 300 on the shaft 34a has a direct relationship to the amount of volume to be retained within the inflation device 30.

In some non-limiting embodiments, the inflation adjustment attachment device 300/shaft 34a includes identifying indicia disposed thereon. The identifying indicia can include, in some non-limiting embodiments, a volume retention indication that substantially corresponds to the amount of volume to be retained within the inflation device 30, and/or the amount of fluid that has been inserted into the balloon.

Still referring to FIGS. 1A-IC and 7, in accordance with one non-limiting aspect of the present disclosure: (i) the inflation device 30 is filled with a maximum (e.g. an upper limit) inflation volume of fluid/gas the user may use during the procedure, as determined from pre-procedure planning (e.g. imaging, electrocardiogram testing, etc.) and patient diagnostic information; (ii) one or more inflation adjustment attachment devices 300 are coupled/attached to one or more holes or apertures 310 disposed along the shaft 34a of the plunger/piston 34 before the housing/chamber 32; (iii) the plunger/piston 34 is advanced until the one or more inflation adjustment attachment devices 300 prevents further advancement of the shaft 34a into the chamber 32, thereby administering a first amount of volume to the balloon 50; and (iv) if an additional amount of volume in the balloon 50 (i.e. additional size/diameter of the prosthetic valve) is warranted, one or more inflation adjustment attachment device 300 are removed from one or more holes or apertures 310 in the shaft 34a, and the plunger/piston 34 can be advanced even further to administer sone or all of the retained amount of volume to the balloon 50. It will be appreciated that any of a variety of additional or alternative steps may be included in a method of using the inflation adjustment attachment device 300. In one non-limiting embodiment, a plurality of inflation adjustment attachment devices 300 are used with the inflation system 10.

It is to be appreciated that the inflation adjustment attachment devices 20, 20′, 100, 200, 300 can be used in combination with one another, can be used independently, and/or can be used in multiplicity (e.g. a plurality of inflation adjustment attachment devices 20 could be used, a plurality of inflation adjustment attachment devices 20 and 20′ could be used, a plurality of inflation adjustment attachment devices 100 could be used, a plurality of inflation adjustment attachment devices 20 and 100 could be used, etc.).

As previously discussed, any of the inflation adjustment attachment devices 20, 20′, 100, 200, 300 can include one or more audible and/or haptic mechanisms that are configured to provide feedback to the user when: (i) the inflation target of the balloon is achieved; (ii) the size/diameter target of prosthetic heart valve is achieved; (iii) the balloon volume is less than the inflation target; (iv) the prosthetic heart valve size/diameter is less than the target; (v) the balloon volume is great than the inflation target; and/or (vi) the prosthetic heart valve size/diameter is greater than the target. It will be understood that the audible and/or haptic mechanisms described with respect to FIGS. 4A and 4B can be included in any of the inflation adjustment attachment devices described herein.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment, or “in an embodiment” in places throughout the specification are not necessarily all referring to the same embodiment. Furthermore, the particular features, structures or characteristics may be combined in any suitable manner in one or more embodiments.

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure, which, as a matter of language, might be said to fall there between. The disclosure has been described with reference to the certain embodiments. These and other modifications of the disclosure will be obvious from the disclosure herein, whereby the foregoing descriptive matter is to be interpreted merely as illustrative of the disclosure and not as a limitation. It is intended to include all such modifications and alterations insofar as they come within the scope of the appended claims.

To aid the Patent Office and any readers of this application and any resulting patent in interpreting the claims appended hereto. Applicant does not intend any of the appended claims or claim elements to invoke 35 U.S.C. 112(f) unless the words “means for” or “step for” are explicitly used in the particular claim.

1. A method for deploying a medical device at a treatment area; said method comprising:
providing an inflation device that is configured to inflate a balloon that correspondingly expands said medical device or to directly expand said medical device;
preparing the inflation device with a predetermined amount of a material to inflate the balloon and/or to expand said medical device;
coupling a first inflation adjustment attachment device to said inflation device, and wherein said first inflation adjustment attachment device is configured to function as a first stop to limit a first amount of said material initially inserted into said balloon or medical device during use of said inflation device;
causing said inflation device to administer said first amount of said material to said balloon or said medical device until said first inflation adjustment attachment device at least partially prevents further material from being administered to said balloon or medical device;
enabling said inflation device to subsequently allow a second amount of said material to be inserted into said balloon or medical device that correspondingly causes further inflation of said balloon or further expansion of said medical device; said step of enabling includes a) uncoupling and removing said first inflation adjustment attachment device from said inflation device and thereafter causing said inflation device to administer a second amount of material into said balloon or medical device to cause further inflation of said balloon or further expansion said medical device, or b) moving said first inflation adjustment attachment device a distance on said inflation device to a new position and thereafter maintaining said first inflation adjustment attachment device on said inflation device at said new position, and wherein movement of said first inflation adjustment attachment device to said new position enables said inflation device to administer said second amount of material into said balloon or medical device after said first inflation adjustment device has been moved to said new position on said inflation device to thereby cause further inflation of said balloon or further expansion of said medical device; and
wherein said second amount of material that is further administered by said inflation device to said balloon or said medical device corresponds to a) a predetermined thickness of said first inflation adjustment attachment device that was previously coupled to said inflation device, or b) said distance that said first inflation adjustment device was moved on said inflation device.

2. The method as defined in claim 1, further providing a second inflation adjustment attachment device that is coupled to said inflation device; and wherein said step of enabling further comprises:
I) uncoupling and removing said second inflation adjustment attachment device from said inflation device and thereafter causing said inflation device to administer an addition amount of material into said balloon or medical device to cause further inflation of said balloon or further expansion of said medical device, or II) moving said second inflation adjustment attachment device a distance on said inflation device to to a new position and thereafter maintaining said second inflation adjustment attachment device on said inflation device at said new position, and wherein movement of said second inflation adjustment attachment device to said new position enables said inflation device to administer said second amount of material into said balloon or medical device after said second inflation adjustment device has been moved to said new position on said inflation device to thereby cause further inflation of said balloon or further expansion of said medical device; and wherein said additional amount of material that is further administered by said inflation device to said balloon or said medical device corresponds to a) a predetermined thickness of said second inflation adjustment attachment device that was previously coupled to said inflation device, or b) said distance that said second inflation adjustment device was moved on said inflation device.

3. The method as defined in claim 2, wherein a) said first inflation adjustment attachment device is uncoupled and removed from said inflation device prior to said second inflation adjustment attachment device being uncoupled and removed from said inflation device, or b) said second inflation adjustment attachment device is uncoupled and removed from said inflation device prior to said first inflation adjustment attachment device being uncoupled and removed from said inflation device.

4. The method as defined in claim 2, wherein said predetermined thickness of said first inflation adjustment attachment device is the same or different from said predetermined thickness of said second inflation adjustment attachment device.

5. The method as defined in claim 1, wherein said inflation device comprises:
a housing that is configured to receive said material; and
a plunger having a shaft, and wherein said plunger is at least partially movably disposed within said housing; and
wherein said first inflation adjustment attachment device is releasably coupled to said shaft or movable along a longitudinal length of said shaft.

6. The method as defined in claim 5, wherein said first inflation adjustment attachment device includes a body having a channel formed therein that is configured to receive at least a portion of said plunger.

7. The method as defined in claim 1, wherein said first inflation adjustment attachment device includes a clip, ring clip, C-clip, or clasp; said first inflation adjustment device includes a tether arrangement, a tab and/or a finger opening used to facilitate in removal of said first inflation adjustment attachment device from said inflation device.

8. The method as defined in claim 5, wherein said first inflation adjustment attachment device includes a body having a channel formed therein that is configured to receive at least a portion of said plunger.

9. The method as defined in claim 8, wherein said second inflation adjustment attachment device includes a clip, ring clip, C-clip, or clasp; said second inflation adjustment device includes a tether arrangement, a tab and/or a finger opening used to facilitate in removal of said second inflation adjustment attachment device from said inflation device.

10. The method as defined in claim 1, wherein said first inflation adjustment attachment device includes an interactive element in communication with a latch, and wherein activation of said interactive element causes said latch to disengage from a portion of said inflation device so that a portion of said inflation device can moved relative to said first inflation adjustment attachment device as said inflation device is moved to said new position on said inflation device, and wherein deactivation of said interactive element causes said first adjustment attachment device to be secured in place at said new position.

11. The method as defined in claim 10, wherein said interactive element is a button, a knob, a switch, a lever, a grip, a controller, a toggle, or combinations thereof.

12. The method as defined in claim 1, wherein said first inflation adjustment attachment device includes one or more audible, visual and/or haptic mechanisms that are configured to provide real-time feedback of said amount of said material delivered to said balloon or medical device.

13. The method as defined in claim 1, wherein said first inflation adjustment attachment device includes identifying indicia, coloring or numbering that provides information about said amount of material that can be further inserted by said inflation device into said balloon or medical device after said first inflation adjustment attachment device is uncoupled from said inflation device or after said first inflation adjustment attachment device has been moved to said new position.

14. The method as defined in claim 1, wherein said material is a liquid or a gas.

15. The method as defined in claim 1, wherein said medical device is a prosthetic valve, stent or angioplasty balloon.

16. An inflation adjustment attachment device for use with an inflation device; said inflation adjustment attachment device comprising:
an arrangement for removably coupling said inflation adjustment attachment device to a plunger of an inflation device and wherein the inflation device is configured to inflate a balloon or medical device; and
a body having predetermined thickness.

17. The inflation adjustment attachment device as defined in claim 16, wherein said arrangement for removably coupling said inflation adjustment attachment device comprises a channel that is configured to temporarily retain said inflation adjustment attachment device on the plunger.

18. The inflation adjustment attachment device as defined in claim 16, wherein said inflation adjustment attachment device includes a clip, ring clip, C-clip, or clasp; said inflation adjustment device includes a tether arrangement, a tab and/or a finger opening used to facilitate in removal of said inflation adjustment attachment device from the inflation device.

19. The inflation adjustment attachment device as defined in claim 16, further including identifying indicia, coloring or numbering that provides information about a volume of material that can be further inserted by the inflation device into sad balloon or medical device after the first inflation adjustment attachment device is uncoupled from the inflation device.

20. An inflation device system that is configured to inflate a ballon or medical device at a treatment area; said method inflation device system comprising:
an inflation device that is configured to inflate said balloon or to expand said medical device; said inflation device includes a housing having a housing cavity that is configured to receive a material; and a plunger having a shaft; said plunger is at least partially movably disposed within said housing cavity; and
a first inflation adjustment attachment device that is connected to said plunger; and
wherein said first inflation adjustment attachment device is configured function as a first stop as said shaft is inserted along a longitudinal length of said housing cavity to limit a first amount of said material to be initially inserted into said balloon or said medical device during said inflation of said ballon or said expansion of medical device; and
wherein removal of said first inflation adjustment attachment device from said shaft or repositioning said first inflation adjustment attachment device along a longitudinal length of said shaft enables said shaft to be further inserted into said housing cavity to insert additional amounts of said material into said inflatable balloon or said medical device.

21. The inflation device system as defined in claim 20, further including a second inflation adjustment attachment device that is connected to said plunger; and
wherein said second inflation adjustment attachment device is configured to function as an alternative or additional stop as said shaft is inserted along said longitudinal length of said housing cavity; and
wherein removal of said second inflation adjustment attachment device from said shaft enables said shaft to be further inserted into said housing cavity to insert additional amounts of said material into said inflatable balloon or said medical device.

22. The inflation device system as defined in claim 21, wherein a predetermined thickness of said first inflation adjustment attachment device is the same or different from a predetermined thickness of said second inflation adjustment attachment device.

23. The inflation device system as defined in claim 20, wherein said first inflation adjustment attachment device includes a clip, ring clip, C-clip, or clasp; said first inflation adjustment device includes a tether arrangement, a tab and/or a finger opening used to facilitate in removal of said first inflation adjustment attachment device from said inflation device.

24. The inflation device system as defined in claim 22, wherein said first and/or said second inflation adjustment attachment device includes a clip, ring clip, C-clip, or clasp; said first and/or said second inflation adjustment device includes a tether arrangement, a tab and/or a finger opening used to facilitate in removal of said first and/or said second inflation adjustment attachment device from said inflation device.

25. The inflation device system as defined in claim 20, wherein said first inflation adjustment attachment device includes a body having a channel formed therein that is configured to receive at least a portion of said shaft of said plunger.

25. The inflation device system as defined in claim 21, wherein said second inflation adjustment attachment device includes a body having a channel formed therein that is configured to receive at least a portion of said shaft of said plunger.

26. The inflation device system as defined in claim 20, wherein said first inflation adjustment attachment device includes an interactive element in communication with a latch, and wherein activation of said interactive element causes said latch to disengage from a portion of said shaft of said plunger so that said plunger can moved relative to said first inflation adjustment attachment device as said plunger is moved in said housing cavity, and wherein deactivation of said interactive element causes said first adjustment attachment device to be secured in place at a new position on said shaft of said plunger.
